(19) United States
(12) Patent Application Publication
(10) Pub. No.: US 2006/0009637 A1
(43) Pub. Date: Jan. 12, 2006

(54) Molecules as produced by endothermic chemical reactions with anhydrous D-ribose

(76) Inventor: Keith Earl Kenyon, Van Nuys, CA (US)

Correspondence Address: KEITH E . KENYON, M.D., 4501 NAGLE AVENUE, SHERMAN OAKS, CA, 91423, US

(21) Appl. No.: 10/886,070

(22) Filed: Jul. 7, 2004

(51) Int. Cl. C08B 37/00; C07H 5/06

(52) U.S. Cl. 536/124; 536/55.3

(57) ABSTRACT

This invention discloses the endothermic reaction that D-ribose undergoes with one or more molecules forming new molecules involving the ribose radical, two such endothermic reactions having been identified as being 1) one mol of D-ribose reacting with one mol of water, resulting in a monohydrate and 2) one mol of ribose reacting with one mol of NaPCA, and finally a mixture of ribose monohydrate and nutrients, herbs, and/or drugs that result in stable edible mixtures of such for consumption.

### RELATED APPLICATIONS

This patent application is related to patent application Ser. No. 09/504,805 and its continuation in part Ser. No. 10/238,064, U.S. Pat. No. 6,553,254 B1, patent application Ser. No. 10/330,566, provisional patent application No. 60/488,914, provisional patent application No. 60/505,102, provisional patent application No. 60/534,272, provisional patent application No. 60/553,725, provisional patent application No. 60/561,489, and provisional patent application No. 60/570,033.

### FIELD OF THE INVENTION

This invention is in the field of nutriceuticals and cosmeceuticals.

### BACKGROUND OF THE INVENTION

This inventor became aware of the availability of D-ribose in commercial quantities in 1999 and began doing research on the molecule as soon as he acquired some. He filed his first patent application Ser. No. 09/504,805 on Feb. 16, 2000. At that time the only patent applications filed or patents issued were for method patents, not molecular, since the ribose molecule itself was in the public domain. Bioenergy, Inc., which was this inventor's supplier, and one of the marketers of ribose, complained that drug companies were not interested in ribose because it was a basic molecule in the public domain. This inventor who had practiced medicine for many years became aware of the healing properties of D-ribose, its use with the failing heart being known to him, as well as its special value for the skin, where its healing properties are significant and not realized by most of whom are skilled in the art. Because all of his patent applications and the one issued patent cited above, were method patents, not involving a new chemical reaction and new molecules, Bioenergy with its own method patents with different objectives, had little interest in his research, resulting in this independent disclosure.

Regardless of who makes it, D-ribose is still a relatively new nutrient on the market and still has the disadvantages that its high manufacturing costs have rendered it too costly for maximum distribution in its present anhydrous powdered or granulated form. Being a sugar with limited sweetness it cannot be marketed readily as a very sweet snack, even with the advantage, that being an end product of the pentose phosphate pathway (PPP), it does not provide calories or food-carbohydrates and does not raise blood sugar. As a result of the low-carb craze, if it could be rendered sweeter like candy, there could be a greater market because it does not raise blood sugar, provide calories or food-carbohydrates. It would have great potential for niche upscale markets in which cost factors are less important than results. In order to understand the role that de novo D-ribose can play in the nutrition of people in general, a partial list of what ribose does includes being a precursor for the following:
1. ATP—(adenosine triphosphate) the body's energy molecule,
2. DNA—the genetic basis for life,
3. RNA—an alternate genetic basis,
4. Glycogen—the storage of the body's energy,
5. NAD—(nicotinamide adenine dinucleotide) the body's fundamental coenzyme,
6. NADH—the reduced form of the basic coenzyme,
7. NADP—(nicotinamide adenine dinucleotide phosphate) contributes phosphorous atoms for energy,
8. NADPH—the reduced form,
9. Leukocyte NADPH oxidase—the enzyme that makes superoxide (O₂⁻), the magic bullet of the immune system,
10. Cyclic AMP (adenosine monophosphate)—a principal hormone second messenger,
11. Glucosamine—needed for joints and connective tissue,
12. NAD-SIR—the gene and its mechanism to cloak all of the genes with protein material to protect them against oxidants that cause aging.

It is obvious that we need ribose and we produce all we can, but we often can't produce enough, especially not enough for #1 above. The fallout is by extracting electrons from food to travel along the respiratory electron transfer chain to molecular oxygen, it takes most of the 72 to 96 hours needed to make ATP, for ribose to be made from glucose in the pentose phosphate pathway (PPP) with the greater opportunity for electrons to be leaked along the way unless the pathway is shortened.

A simplified version of the known chemistry of D-ribose in the PPP is as follows: glucose+protein enzymes and coenzymes including NAD(P)+electrons→glucose-6-phosphate+more enzymes+more electrons→6-phosphogluconate+more enzymes and electrons and with NAD derivatives used through the PPP tediously removes a carbon atom from the glucose, it to become carbon dioxide mostly and by doing all of the foregoing, leaks many free electrons in order to obtain the most vital molecular segment in the body, a 5-carbon-atom sugar radical—ribose—which has been phosphorylated in the PPP process→ribulose-5-phosphate→ribose-5-phosphate→5-phosphoribosyl-1-pyrophosphate+(ultimately) adenine+protein enzymes+more phosphate+enzymes including NAD(P)+electrons→AMP (adenosine monophosphate)→ADP (adenosine diphosphate and (ultimately) ATP-energy. This synthesis takes 72 hours or more and considerable energy because the mitochondria must remove that carbon atom from 6-carbon-atom glucose to make 5-carbon-atom ribose. The in vivo endothermic reactions involved here may be like in vitro chemical reactions for production of such molecules or new molecules containing D-ribose for the purposes of marketing them as supplements or drugs, as is the purpose of this disclosure.

It is obvious that many electrons would not be leaked to form superoxide in the fatty layers of the cells, and much time to make ATP would be saved if most of this pathway were avoided. D-ribose, if taken, can greatly shorten the PPP: D-ribose+protein enzymes and NAD(P)+electrons→ribose-5-phosphate→5-phosphoribosyl-1-pyrophosphate+(ultimately) adenine+protein enzymes+more phosphate+enzymes including NAD(P)+electrons→AMP→ADP and (ultimately), ATP-energy with up to 90% fewer leaked electrons capable of forming superoxide with oxygen in addition to much faster-formed ATP. All of the previous patent applications and patents concern the chemical reactions of de novo D-ribose formed in the PPP and involving the use or method patents and applications using anhydrous solid D-ribose. This disclosure will seek to revolutionize this concept.

This invention is designed, among other things, to overcome the deficiencies of previous applications and inventions by disclosing new molecular forms employing the D-ribose radical by a newly understood property of D-ribose to enable it to make new substances under control.

### BRIEF SUMMARY OF THE INVENTION

This disclosure seeks to use anhydrous solid D-ribose to make one or more new molecules. The primary molecule formed by this disclosure is very sweet, actually sweeter than glucose, sucrose, or anhydrous D-ribose before such ribose reacts with a separate molecule, HOH, to yield an endothermic chemical reaction. Whereas, exothermic reactions are commonplace, endothermic reactions are relatively rare. Exothermic reactions increase entropy considerably while endothermic reactions are more balanced. A common commercial endothermic reaction is ammonium nitrate reacting with water and producing ammonia and nitric oxide. This reaction forms the most common cold pack. In an endothermic chemical reaction more chemical energy is stored in the bonds of the produced molecules than was in reactant molecules. Because of the law of concentration of energy, this increased energy in reactant molecules makes their resulting combination very cold.

Water one way or another appears to be involved with common endothermic reactions and often these involve ammonium nitrate, a fertilizer and an element of some explosive materials, the most profound kind of exothermic reactions. In all kinds of endothermic and exothermic chemical reactions the reactants are different molecules than what are produced. In the case of using ammonium nitrate with something besides pure water, barium hydroxide octahydrate is cited. Water is still provided; in this case eight water molecules are attached to one barium hydroxide molecule. Thus the molecular weight of this molecule is composed of 148 parts hydrogen and oxygen (H₂O), while 137 parts are provided by barium, nearly 50/50. In this reaction two ammonium nitrate molecules are also required resulting in barium nitrate and ammonia being formed as well as water unattached to any other molecule Barium hydroxide and barium nitrate are both very toxic. Once again the reaction becomes cold.

On the other hand, two different chemicals can be joined together in the case of fluids without an exothermic or endothermic reaction. This is the case of substances with which an ordinary solution can be formed. A common example is for certain solid salts like sodium chloride to dissolve in a liquid like water. In the case of a sugar like glucose, fructose, or sucrose, once again a water solution can be made without new substances being formed or much change in entropy by these solutions. Ammonium nitrate can form a solution but also has endothermic as well as exothermic properties, the endothermic reactants involving either water or the water attached to another molecule.

In the case of the special sugar D-ribose we have a difference in that, on a molecular basis, one molecule of D-ribose, reacting with one molecule of water directly multiplied by a one-on-one basis, only an endothermic reaction occurs. In the case of glucose (molecular weight 180), when adding multiples of a mol of glucose to the exact same number of mols of water (molecular weight 18), the sugar remains the same but becomes lumpy in contrast to when one mol of ribose (molecular weight 150) is substituted for each mol of glucose. Here a profound endothermic reaction occurs with ribose substituted, and the resulting product is far sweeter, denser, and homogeneous, forming an amber paste. The fact that this product is much sweeter than solid anhydrous D-ribose was disclosed in provisional patent application No. 60/505,102 “D-ribose Made Sweeter than the Natural Solid” which was followed up by provisional patent No. 60/534,272 about the endothermic nature of the sweetening. The product so achieved became much more soluble in water than glucose. Further research disclosed the fact that D-ribose was endothermic with respect to other molecules also, resulting in provisional patent application No. 60/570,033 being filed.

The fact that D-ribose is capable of endothermic reactions has not been disclosed prior to this disclosure and its previous provisional patent applications. Since endothermic chemical reactions are chemical reactions, different molecules than the reactants result. There is more total chemical energy in the new bonds than there were in the reactants, but overall the initial entropy is maintained in that energy is not created only transferred. Since ribose is a key sugar in the body, the fact that it is endothermic must play a prominent role in its involvement in the PPP and with other reactions. This disclosure enables ribose's energy bonds to be increased by forming new molecules. Whether a new molecule is formed depends on whether or not ribose can act endothermically with a substance not itself. The nature of the new molecules that are formed depends on what molecule can enable ribose to create an endothermic chemical reaction.

Most liquid molecules that contain only carbon, hydrogen, and oxygen in saturated bonds are not endothermic with ribose. These would include some simple ones like ethylene glycol and glycerol. Solid molecules do not appear to act endothermically with ribose. At the time this disclosure is being submitted, research is being done to identify more molecules that act endothermically with ribose. One such molecule, a liquid, the sodium salt of pyrrolidone carboxylic acid, has been disclosed in provisional patent application No. 60/570,033. In this case the molecular weights of the two reactant molecules are more equal, and since both are anhydrous, water is not involved as an independent reactant molecule but may be formed as a reaction result. A reaction either with the nitrogen bond in the pyrrole radical or a bond created by the change of the carboxyl radical in removing a water molecule, commonly referred to as forming an ester, are two possibilities.

Regardless of which, the point is that no other sugar does this at room temperature with ambient air pressure. Furthermore, although NaPCA is not sweet, the product of the endothermic reaction is, but not to the level that the endothermic reaction of D-ribose and water reaches. The viscosity of the NaPCA-ribose reaction is high, just as is the viscosity of the H₂O-ribose product, and the solubility of both in water is exceptionally high.

On the other hand, other liquid carboxylic acids may or may not react with ribose in an endothermic reaction and have not been investigated at the time this disclosure is being submitted. One of these, butyric acid, is a foul-smelling ester of glycerol. Glycerol itself does not enable ribose to enter into an endothermic reaction, Nevertheless the possible reaction of ribose with butyric acid will be ultimately investigated along with its isomer, isobutyric acid, with different physical properties but similar chemical properties to butyric acid. Other endothermic reactions with ribose may occur, but the toxicity of all resultant chemicals, including environmental adversity, must be tested as well as their uses.

The features of the present invention which are believed to be novel are set forth with particularity. The present invention, both as to its organization and the manner of operation, together with the further objects and advantages thereof, may be best understood by reference to the following exemplary and non-limiting detailed description of the invention, wherein;

### DETAILED DESCRIPTION OF THE INVENTION

### NO DRAWINGS

The preferred embodiment is to enable pure anhydrous D-ribose to react in an endothermic chemical reaction with a second molecule and get a product that is neither the original D-ribose nor the second molecule. This type of reaction is possible at room temperature and ambient air pressure because of the endothermic nature of D-ribose. The resulting molecule, in the case of one mol of D-ribose reacting with one mol of water, is nontoxic when taken internally, but all other such endothermic reactions may have various degrees of toxicity depending on the nature of the resulting product. Neither the ribose-water reaction nor the ribose-NaPCA reaction results in a well known molecule but are new ones in both cases. We should expect all other ambient endothermic reactions with D-ribose and a second molecule, to result in at least one new molecule previously unknown in vitro but may be made naturally in vivo within the living body and identified there as a result.

As stated above, the preferred embodiment is to react one mol of anhydrous solid D-ribose with one mol of a second molecule that is capable of reacting with said D-ribose in an endothermic chemical reaction. So far the direct endothermic reaction of D-ribose is with liquids and not solids. D-ribose does not react endothermically with other sugars or with almost all water soluble solids such as ascorbic acid, niacin, L-arginine, etc. The use of water-insoluble molecules like creatine in this endothermic reaction will be discussed below.

A specific example of making the monohydrate of D-ribose as an endothermically-produced product in commercial amounts would be to have one mol of D-ribose react with one mol of H₂O in identical multiples of each. Doing such in this example is one kilogram of anhydrous D-ribose and 120 milligrams of pure water being mixed together. This will result in 1.12 kilograms of a product that is neither like anhydrous D-ribose nor water, but is quite soluble in water. The resulting product would be expected to be the monohydrate of D-ribose. Ribose is ordinarily regarded as a saturated 5-carbon atom ring molecule with one hydroxyl group per carbon atom, but is also written as HCHOH—CHOH—CHOH—CHOH—HC═O. One mol of water changes one mol of ribose by this endothermic reaction into an entirely new molecule. Two mols of water first change the one mol of ribose available to be hydrated into a monohydrate. The second mol of water takes the step of forming a concentrated aqueous solution of the newly hydrated molecule. As a result we can safely call the new molecule a ribose monohydrate. As additional mols of water are added the solution becomes less viscous but still very sweet and becomes increasingly translucent after the weights of ribose and water become equal and then unequal in favor of more water. To which carbon atom does this new water molecule attach? The number-5 (last carbon atom) location shown above is most likely.

In the case of niacin, a molecule that is soluble in water, when mixing equal amounts of powdered niacin and anhydrous powdered D-ribose, nothing remarkable happens. It still remains a powdered mixture, but if then a small amount of water is added to the mixture, an endothermic reaction occurs with the water and the ribose. The mixture now turns cold and an opaque paste is formed that does not have the same amber color of when the monohydrate is formed separately with niacin being 50% of the mixture, not so with smaller amounts. Similar reactions occur with other molecules including amino acids, vitamins, and minerals. The intense sweetness of ribose monohydrate would obscure the bitterness of some of these nutrients, soluble or insoluble, and the endothermically-derived stronger bonded monohydrate should be more stable than the anhydrous form of D-ribose when mixed in vitro. A paste forms with soluble niacin, L-arginine, etc., or a powder with some insoluble molecules.

The natural amber color of the monohydrate of D-ribose will be altered by the strength and color of the respective ingredients added to it. Each molecule or nutrient must be investigated with respect to its physical composition when the reaction forming ribose monohydrate occurs and the results investigated as to how clinically effective for various conditions it is compared to the nutrients when administered separately. A mixture valuable for the heart is the combination of 2 grams of L-arginine and 5 grams of anhydrous D-ribose thoroughly mixed together first, then the resultant mixed with 0.6 gram of water which results in a good-tasting, soluble, amber paste.

Therefore, it can be assumed that ordinarily D-ribose will not react endothermically with molecules that are in the solid state but a water-soluble paste-like mixture can ensue if the monohydrate is made by such endothermic means. Endothermic reactions with solid molecules do occur. The example of barium octahydrate reacting with ammonium nitrate, two solids, does result in an endothermic reaction, but in this case one of the solids, barium hydroxide, has water molecules attached to it, so it is toxic barium hydroxide hydrate forming toxic barium nitrate unlike nontoxic ribose forming a ribose hydrate.

Another example of the preferred embodiment does not have water on the reactant side of the chemical equation. It is to take the molecular weight of anhydrous D-ribose and react it with the molecular weight of the anhydrous molecule known as the sodium salt of pyrrolidone carboxylic acid in multiples of their respective molecular weights. This reaction results in an amber viscous fluid that has increased viscosity over the colorless NaPCA. A molecule of water or a hydrate thereof may be part of the resultant product.

These two examples disclose the use of either water or a substance very soluble in water. Consequently not having the multiples of a mol of one exactly equal to the other, still will make the resulting product useful. Since all these reactant substances are soluble in water as are the resulting products, small inaccuracies in putting the reactants together can be used either as they come out of the reaction, or the resultant product can be diluted with water and thereby also make a very useful product. The exception to this will be discussed below when aqueous insoluble molecules are presented.

In the case of the endothermically-created monohydrate of D-ribose, provisional patent application No. 60/505,102 “D-ribose Made Sweeter than the Natural Solid”, discloses the then preferred embodiment to be 50% D-ribose and 50% water by weight. This would result in one mol of ribose monohydrate and about 7⅓ mols of water on the product side of the chemical equation, still quite a sweet product. Nevertheless, this is not as sweet as the pure viscous endothermically-derived molecule where the weight of the ribose is 8.333 times the weight of the water. When water is added to ribose, all the water added after 1/8.333 of its weight compared to that of ribose, the ribose monohydrate formed just becomes a further dilution of the aqueous solution of the ribose monohydrate. As more water is added ultimately the sweetness of anhydrous solid D-ribose will become greater than the dilute solution of the originally very sweet ribose monohydrate, originally an opaque very viscous substance of about double the density of anhydrous D-ribose. At some point the successive dilutions will render the solution far less sweet to the average taste than the anhydrous solid. When very dilute solutions are made, this disclosure will cease to govern such dilutions with respect to D-ribose, because the prior art has virtually non-sweet dilutions. When dilutions are far less sweet than the natural solid anhydrous molecule and far less sweet than its monohydrate, they are the way ribose and water are used today. With these products 10 grams or less of anhydrous D-ribose are put into 300 milliliters or more of water. Such substantial dilutions are in the public domain, but concentrated solutions are new art since they have different physical properties than either anhydrous solid D-ribose or its very dilute solutions.

Therefore, the initial product of the endothermic chemical reaction of D-ribose and water on a mol-to-mol basis is an amber very viscous opaque fluid that could be described as a paste. Add water slowly to this monohydrate molecule, and a gradual physical change comes to the substance leading up to a clear, translucent viscous amber solution. When less than ½ mol of water is added to the mol of ribose monohydrate, it becomes more fluid but still viscous. Even with such a small amount of additional water, over a period of time, the product will become a thick amber clear translucent solution that points out the incredible solubility of D-ribose in water. By weight a kilogram of anhydrous D-ribose can be dissolved in less than 168 grams of water and if enough time is allowed a clear translucent amber viscous solution emerges at some point before as much as 168 grams total water have been used. The monohydrate can also be made first followed by the rest of the water to turn the opaque amber paste into the viscous concentrated clear amber translucent solution. By the time an equal amount of water by weight is added to the weight of the original anhydrous D-ribose, the viscosity has been reduced and with it the sweetness, but it still is quite sweet, the progression towards less viscosity and sweetness occurring on a fairly uniform basis. By the time the dilution gets to how Bioenergy, Inc., an entity with obvious ordinary skill in this art, discloses its maximum concentration of anhydrous D-ribose, as 10 grams of anhydrous D-ribose powder put into 300 milliliters of water, there is only a hint of sweetness, and further dilutions taste nearly the same as the water they are mixed into. To market D-ribose for the heart originally, Bioenergy used dextrose also to sweeten arginine when diluted, not necessary when ribose monohydrate is combined with it.

Since the monohydrate of D-ribose has virtually no toxicity and is of similar consistency and amber color as honey, although not as translucently clear as honey but of complementary taste, putting the two substances together, such as in equal amounts by weight, results in an amber translucent, very sweet mixture of a similar viscosity but with less translucence than honey. The combination on a gram-for-gram basis, while more expensive than the honey is less expensive than pure ribose monohydrate and is tasty enough to be a snack at present for upscale people with half the calories and food-carbohydrates of honey. It can also be a sweetener for food and tea.

In the case of the endothermic reaction between ribose and NaPCA, being a cosmeceutical rather than a nutriceutical, sweetness does not play a role. Nevertheless, as a solid, D-ribose is the endothermic molecule that governs dilutions. Since anhydrous D-ribose is a solid and NaPCA a liquid, if excess mols of one are used undiluted it is desirable for the excess mols to be NaPCA so it will stay a viscous liquid. NaPCA has been marketed as a dilute solution as has D-ribose, but not the two together. We thus have several ways to use the endothermic reaction capability of ribose in this situation. The first is when water is the solvent of NaPCA and not a reactant. The second is when the monohydrate of ribose, formed endothermically, becomes part of the solution with NaPCA. A third is when the endothermically-created NaPCA-ribose molecule is formed, and it can be directly dissolved in water. In each case the resulting product or products can be mixed with most cosmeceuticals.

Thus if NaPCA is added to water independently of the ribose and then ribose is added, the ribose becomes the monohydrate with respect to one mol of ribose and one mol of water in an endothermic reaction that is not noticed in gross physical terms as the water obviously becoming colder to the touch, because the endothermic reaction is so small in terms of volume of the solvent. So we now have a choice of having either a solution of NaPCA-ribose or NaPCA and ribose-monohydrate. Both combinations are effective, and the question of which one is more effective, if one turns out to be so, has not yet been determined. Furthermore, either one can be made part of other topical products. The cost of manufacture is essentially the same since it costs little more to mix the NaPCA and ribose together first and then put the product into solution than to put the anhydrous powder of D-ribose into water followed by the viscous liquid NaPCA. There is still an endothermic reaction whether a mol of water attaches to a mol of ribose or a mol of NaPCA attaches to a mol of ribose. Both combinations are soluble in water. If more ribose than NaPCA is added to extra water, then the ribose monohydrate will be in greater amount than the NaPCA. With the reverse there will be more NaPCA than ribose monohydrate. Either way the resulting solution will still work well, can be added to the same cosmetics or topical skin products, and cost effectiveness is the only variable that needs attention.

With respect to using D-ribose with other substances with which it can form an endothermic reaction other than with water, some (other than ribose) may have toxic properties to begin with. There is no reason to expect that the non-toxicity of ribose will make the product nontoxic also, and such new molecules must be thoroughly tested both for toxicity to living beings and to the environment before they are marketed. A case in point is the proposed reaction of D-ribose and the simple fatty acid butyric acid which like NaPCA is a liquid carboxylic acid. If an endothermic reaction takes place a new ester may be formed if chemically possible such as CH₃—CH₂—CH₂—O—C—O—CH₂—CHOH—CHOH—CHOH—CHOH. The sodium salt of a carboxylic acid formed with a pyrole radical, does provide a carboxylic acid unesterfied, possibly resulting in an ester. Perhaps a carboxylic acid already an ester may not form a double ester even if a sodium atom is added to make a sodium salt of butyric acid as a reactant with ribose. Such possible reactions will be tried now that the endothermic nature of D-ribose has been discovered.

Other liquid carboxylic acids that are not esters can be represented as an example here by acetic acid. Pure acetic acid may or may not react with ribose in an endothermic chemical reaction to form a possible ester, which possible molecule, ribose combined with acetate, would probably be soluble in water and may or may not have medical or industrial uses. Once again if formed its toxicity requires to be determined as well as its environmental impact. Other possible endothermic reactions with ribose will be derived by trial and error initially, but if it is an endothermic reaction this disclosure seeks to embody it.

Anhydrous D-ribose is an unstable molecule in vitro when added to certain other molecules. It can react with many substances, so putting it into a mixture with other molecules such has been mentioned in the prior art can result in deterioration of the resultant mixture. Bioenergy has found that in the presence of the amino acid L-arginine and dextrose, a ribose heart product, Corvalen, became degraded, and it had to be changed to the anhydrous ribose powder alone. This disclosure has pointed out the endothermic nature of D-ribose. Obviously the monohydrate of ribose in vitro having gained energy in its bonds by the endothermic reaction with ribose, may be a much more stable molecule in vitro than the less energetic anhydrous form. On the other hand, if more reactive, it's still a new molecule and will have a different positive value than its anhydrous precursor. D-ribose monohydrate can be stored by itself if highly reactive, but we are assuming it will not react and degrade when stored with amino acids and other solid nutrients including creatine monohydrate. When anhydrous D-ribose is thoroughly mixed with L-arginine such as ⅓ arginine and ⅔ anhydrous powdered D-ribose, the amber uniform smooth opaque paste-like product is soluble in water and has an acceptable taste. Of course, if too much arginine is used, the taste deteriorates.

Ribose monohydrate can mix with other aqueous insoluble solids even bitter nutrients like many vitamins and minerals are. If a bitter nutrient is mixed with a concentrated water solution of D-ribose monohydrate or the pure monohydrate itself, this viscous sweet substance not only will enable the nutrients to be absorbed by the body quickly, but they will taste well enough to be tolerable in this form. The effectiveness of this part of the disclosure was shown when the inventor combined two grams of niacin with five grams of the ribose monohydrate of this disclosure thoroughly mixed, this time being amber with less niacin added. He then tried the resultant mixture, putting about 500 milligrams of niacin from this mixture into his mouth and the taste of two parts of niacin in five parts of ribose monohydrate was very compatible. Then within one minute of the ingestion of this anhydrous mix, he became very hot and his face became flushed to the point it became beet red in color, a typical reaction when taking plain niacin in such a dosage, but showing that this anhydrous mixture is also absorbed very fast.

In using anhydrous D-ribose with an insoluble nutrient, such as creatine, the easiest way to make such a product is to blend the anhydrous powder of D-ribose with powders of one or more solid anhydrous nutrients into whatever portions are deemed to be desired. This can include just a solitary nutrient as in the case of niacin, just described, or it can be a mixture of solid nutrients, water soluble and/or insoluble. The weight of bitter nutrients mixed with anhydrous D-ribose should be less than half the weight of the ribose in order to make the resulting taste as compatible as possible. Then for each mol of ribose in the mixture of the powders, a mol of water is added and the resultant complex thoroughly mixed together. The endothermic reaction of ribose forming the monohydrate will quickly be noticed, and a paste will result that has a tangy sweet taste depending on the bitterness of the nutrients and their amounts that were mixed in the first place.

Much is learned by mixing water with anhydrous D-ribose and a substance insoluble in water but also unstable in water like creatine monohydrate. New lessons in aqueous solubility can be learned and combining two nutrients ordinarily unstable when mixed, now have stability. Creatine monohydrate is not bitter but still has a disagreeable taste. If ribose monohydrate is formed first and then mixed with the creatine monohydrate in equal portions by weight, the two monohydrates form a soft solid. This solid can be consumed as one would a piece of cheese or teaspoon of peanut butter. It has a rather sweet taste, is palatable, and can be wrapped like candy. If it is powdered there is an element of insolubility in a dilute aqueous solution. On the other hand, by mixing powdered anhydrous D-ribose with an equal amount of creatine monohydrade thoroughly, then slowly adding water in 1/8.333 of the weight of the ribose, an amber powdered product can be the result by grinding. Thus, a blend of ribose monohydrate and creatine monohydrate forms a product that has its own physical properties that are different from the two monohydrates separately, and depending on the way they are mixed actually, results in different forms with different solubility factors.

None of the mixtures are supremely soluble in water as is ribose monohydrate but are also not as insoluble as creatine monohydrate itself. For example, when the paste is powdered and put into water, a precipitate forms. The affluent liquid above the combination precipitant has a more palatable taste than the affluent liquid above the creatine monohydrate alone when subjected to the same treatment. The fact that the ribose monohydrate is very soluble in water and the creatine monohydrate is not, shows up when this combination is put into water. On the other hand, when the two anhydrous powders are mixed thoroughly and then water added slowly to the mixture and again thoroughly mixed, a physically different combination is formed. When such a powdered blend is placed in water, it is far more soluble than the creatine by itself. Creatine monohydrate should not be packaged with anhydrous-powdered D-ribose, because such a mixture threatens the stability of both in vitro. Creatine being a carboxylic acid probably reacts with the unprotected No. 5 carbon atom with its double bonded carbon-oxygen radical attached to the molecule from the linear perspective. When these mixtures become composed in part by ribose as a new molecule with higher energy bonds, it appears that it should be more stable when mixed with creatine monohydrate. Such appears to be the case with amino acids like L-arginine also, and all can be so marketed. Instructions for creatine monohydrate are to put it in water or juice and drink soon thereafter. Ribose monohydrate offers a way to ingest creatine monohydrate without having to put it into a drink.

When one considers that creatine is a nitrogen-heavy carboxylic acid, H₂N—C(NH)—NCH₃—CH₂—COOH, it is little wonder that when anhydrous D-ribose is mixed with it and stored, the resulting mixture degrades. Creatine, a solid, does not itself react with ribose in an endothermic reaction such as the nitrogen-containing liquid carboxylic acid, NaPCA, does, but D-ribose monohydrate obviously has an affect on creatine monohydrate by making a mixture with different properties and greater stability than was realized prior to this disclosure. The molecule of water will attach to the reactive carbon hydroxyl bond of ribose and then, now having a much stronger bond, requires more energy to destabilize it. Of course, such energy is readily supplied by the living body in vivo once ingested, but until ingested a strong bond should be less subject to molecular reaction when stored in vitro with molecules it would otherwise slowly react with. Since anhydrous ribose and anhydrous creatine monohydrate react with each other, care must be taken to be sure that the exact amount of necessary water is added to exact amounts of anhydrous D-ribose and creatine monophosphate when mixed together so that there will not be a significant excess of any of the three molecules enabling them to each react on a basis of a molecule-to-molecule-to-molecule or multiples of each thereof for maximum stability, solubility, and standardization of the mixture.

One more point, it has been reported that creatine when used on people with heart failure, the ejection fraction did not improve but skeletal muscle action did, resulting in increasing both strength and endurance. Since taking ribose alone improves cardiac diastolic function and probably systole also, plus its role with skeletal muscles, synthesizing ATP faster and increasing ATP salvage, this combination will be of value for the patient in heart failure. This technique used with more soluble salts of creatine such as di-creatine citrate or even creatine phosphate results in a soluble ribose-creatine mixture, whichever one it is. This is followed by mixing a mol of water per mol of anhydrous D-ribose present in the mix or even making the ribose monohydrate first and then mixing it with whatever creatine molecule is used separately into the presenting mix, the resulting product will have different aqueous solubility characteristics depending on which creatine molecule or mixture of creatine molecules is used. In either mixing scenario more than one molecule of creatine can be used as long as the same number of mols of ribose and of water are mixed together at some point in the mixing process whether as the first step, intermediate step, or the last step. If the most soluble creatine salt, di-creatine citrate, is used, we can anticipate that it will be even more stable with respect to crystallization at 4° C. than the monohydrate-ribose monohydrate mix which is soluble at room temperature and body temperature but has some crystalization at 4° C. when equal parts by weight of creatine and ribose are mixed or in other words, unequal mols of the two.

In the case of insoluble creatine monohydrate, when direct multiples in grams of the molecular weight of anhydrous creatine (131) but in the form of creatine monohydrate so multiples of 131 of creatine monohydrate are mixed with direct multiples in grams of the molecular weight of anhydrous D-ribose (150) or multiples of 150, and then direct multiples of the molecular weight of water (18) carefully mixed in, the resulting mixture dissolved in water in soluble amounts at room temperature, does not crystallize at 4° C. Therefore, it must be concluded that, while unequal molecular proportions by weight of creatine and ribose can be mixed, to ensure maximum solubility there should be less weight of creatine monohydrate than ribose to make a more soluble mix. The endothermic reaction of D-ribose enables this to happen so that the water molecule only attaches to the ribose molecule. A less soluble paste can be made by putting the water into the mix all at once, and this is the most inexpensive way to mix the ingredients. If the water is introduced very slowly even spraying in the allotted water and mixed while it is being added, little amber globules will appear within the powdered mix as the water is added. This is due to the fact that only the ribose attracts the water. When the mol of water with respect to the mol of anhydrous D-ribose has reached the end, the entire mix needs to be mixed further. A soluble powder in a dilute aqueous solution will emerge. From the clinical point of view all of the creatine monohydrate-ribose monohydrate mixes will work well on the heart in vivo. The most stable kind of such mix in vitro must await further research, but since every mix tastes good, compaction into edible tablets, even separately wrapped like a candy, is possible for maximum convenience to the user. In this case equal amounts by weight of ribose monohydrate and creatine monohydrate can be used because a solution does not enter into the consumption of a chewable tablet. L-arginine also makes such a tablet.

When a fairly water-soluble molecule like ascorbic acid, arginine, or dicreatine citrate, is used, since ribose is chemically reactive with water by an endothermic reaction on a mol-by-mol basis and these not, the water molecules will attach to the ribose molecules before such water can form a solution with a fairly soluble nutrient like L-arginine. Since the ribose monohydrate is also much more soluble in water than L-arginine, additional water added will form a solution here and with many such mixtures. It must be kept in mind that such soluble molecules besides L-arginine also do not react with water like ribose does. They only dissolve in it like glucose. On the other hand, when water is added to ribose containing a soluble mixture, water will react with the ribose endothermically until the ribose is used up and then an ordinary solution ensues of the entire mixture with the ribose now being in its very soluble monohydrate form. Such tastes good and is compactable.

Once again, if D-ribose is going to be stored with other substances like amino acids with their carboxyl groups, vitamins, and minerals, for maximum stability, the ribose monohydrate should be an anhydrous monohydrate. This is not an oxymoron. It simply means that if the presence of ordinary water threatens the stability of the resulting mixture, there cannot be extra ribose or extra water associated with the ribose monohydrate. This is the case with substances that degrade in the presence of excess water like creatine monohydrate, in this case converted to poorly absorbed molecular creatine. Whereas, both ribose and creatine are synthesized in the body and used to facilitate energy production in the muscles, ribose is very soluble in vitro and creatine very insoluble.

On the other hand, if extra water does not interfere with a resulting mixture, merely forming a stable solution, then the mixture can be an aqueous solution or if some parts are insoluble, a suspension. If the amino acids and vitamins are bitter, a very concentrated ribose solution will need to be formed in the proportions so that the intense sweetness of the ribose monohydrate will predominate in the taste. By first mixing completely a given amount of powdered anhydrous D-ribose with a given amount of one or more powdered anhydrous soluble and sometimes insoluble nutrients, drugs, and herbs, a thorough mix can be achieved. Then if one mol of water or 1/8.333 by weight of the weight of ribose so mixed and there is not excess of the additional substance, a uniform paste can be achieved in some cases, a powder in others, and the way the water is mixed in can influence the product as disclosed above. Although not necessary when the paste is consumed, if a liquid is desired, care must be taken to prevent precipitation of the separately insoluble parts when the mixture so achieved is placed in additional water and the mol-by-mol combination needs solubility in water.

All of the method patent applications cited under “Related Applications” above can now be re-applied for using the molecule D-ribose monohydrate in place of the anhydrous D-ribose term. Of particular interest with respect to this is patent application No. 10/330,566. Here the use of anhydrous D-ribose as a homeopathic carrier was disclosed and no prior art was cited against this homeopathic-carrier contention. Ribose as a chemical is more reactive than lactose, the principal homeopathic carrier, and as a consequence may not be as desirable as a carrier for that reason. On the other hand, made into the monohydrate, its increased energy bonds should render it to be more stable until it enters the body where with the energy there it can be altered as the body requires. We would also expect that the strong endothermically-created bond of ribose monohydrate will not succumb as readily to deterioration in vitro with substances other than homeopathic.

If the homeopathic drugs are present in 6× dilutions, it would be one part in a million. If such a dilution in a small amount were added to a tablet of lactose or sucrose, it would be marketed as part of the solid lactose or sucrose. D-ribose monohydrate can serve as a surrogate for other sugars by compaction and then made into amber colored tablets. The homeopathic drugs can be added to the ribose monohydrate in the same way they are added to lactose tablets. The fact that homeopathic drugs are considered to be non-molecular energy makes their addition to any sugar, including the monohydrate of ribose, easier.

Drugs other than homeopathic, or to contrast the term, “homeopathic”, allopathic drugs, can be mixed directly into the ribose monohydrate paste, or the drug in powdered form thoroughly mixed with anhydrous powdered D-ribose, and then the resultant mixture further mixed with water in 1/8.333 the amount by weight of the anhydrous D-ribose strength. Allopathic drugs unlike homeopathic are not used in extreme dilutions but in full strength. Therefore, for taste purposes it would be preferable to limit the amount of such drug when quite bitter, to be half or less of the monohydrate of D-ribose's weight mixed in.

Such a molecular mix may render the combination more absorbable by any route as a result of the carrying capacity of the D-ribose monohydrate as is the case of creatine monohydrate. Routes would include the mucous membranes of the anus and rectum, the mouth, the veins or arteries, and even the skin. Of course, having a relatively expensive carrier for inexpensive molecules that absorb adequately already, this procedure may not be cost effective. On the other hand, when a molecule is expensive and it is proven that incorporating it with D-ribose monohydrate via the endothermic chemical reaction of this disclosure is water soluble, it would be especially valuable when rapidity of absorption is important, such as with antibiotics. It would also be valuable when it is desired to administer amino acids intravenously with the ATP precursor, ribose. If such could be rendered soluble by the addition of the energy nutrient ribose monohydrate as the carrier, making them mutually soluble in aqueous solutions, of which the blood is a prime example, then a superior, cost-effective medical product could evolve using this endothermic reaction. Not every insoluble molecule will respond to this procedure but many will, and even insoluble mixes may still absorb better by some of these routes. This would include using refined herbs as well as drugs.

A special case may be made with respect to the intravenous route. For several years Bioenergy, Inc. has attempted to get FDA approval for an IV solution of D-ribose. In keeping with glucose it should ordinarily be provided as a 5% solution, because more concentrated solutions tend to put the veins at risk. Bioenergy was unaware that when ribose is added to 20 volumes of water an endothermic reaction was taking place because the 20 times the volume of water masks the reaction. The same is true when Corvalen tells its customers to put five grams of its D-ribose in a glass of water. No one would think of putting five grams of water into 100 grams of ribose and witness the endothermic chemical reaction that would ensue. Nevertheless, now we know about it and using the forgoing and other such mixtures for intravenous use may have medical applications beyond just having ribose there.

While we have discussed this disclosure as it applies to human beings, the same endothermic chemical reactions using anhydrous solid D-ribose applies to other animals and products designed for them.

Finally, while we have used the common term, ribose, in this disclosure, it is not limited to ribose, and includes any 5-carbon precursor or substitute of ribose, including D-ribose, ribulose, xylitol and xyulose if they can be a reactant in an endothermic chemical reaction with at least one other molecule, forming one or more new molecules.

While particular variations of the present invention have been described, it will be obvious to those skilled in the art that changes and modifications may be made without departing from my invention in its broader aspects of producing endothermic reactions with D-ribose and other molecules.

1. An endothermic chemical reaction employing the five-carbon atom pentose usually referred to as ribose and any other similar molecule that will engage in such a reaction forming one or more new molecules:

2. The reaction according to claim 1 in which D-ribose is the form of the ribose used.

3. The reaction according to claim 1 in which water is one of the chemical reactants.

4. The reaction according to claim 1 in which NaPCA is one of the chemical reactants.

5. The reaction according to claim 1 in which other molecules can be one of the chemical reactants.

6. The reaction according to claim 1 in which the resultant molecule or molecules can be dissolved in water.

7. The reaction according to claim 1 in which the resultant molecule or molecules can be mixed with other molecules not part of the reaction.

8. The reaction according to claim 1 in which the ribose is anhydrous.

9. The reaction according to claim —1 in which a useful product will be recovered when the mols of ribose are not equal to the mols of the other molecule on the reactant side of the chemical equation.

10. The reaction according to claim 1 in which said product molecule is a water combination with said ribose and said molecule can be used as a carrier for homeopathic drugs.

11. The reaction according to claim 1 in which said product molecule is a water combination with said ribose, said molecule can be used as a carrier for other than homeopathic medicaments.

12. An endothermic chemical reaction between the five-carbon atom dextrorotary pentose referred to as D-ribose and water in which a new molecule is formed.

13. The reaction according to claim 1 in which one mole of water and one mole of D-ribose are mixed.

14. The reaction according to claim 1 in which the molecule formed is a monohydrate.

15. The resulting reaction according to claim 1 in which the new molecule forms at room temperature.

16. The reaction according to claim 1 in which the new molecule is amorphous.

17. The reaction according to claim 1 in which the resultant molecule is light brown in color.

18. The reaction according to claim 1 in which the resultant molecule melts at body temperature, 37° C.

19. The reaction according to claim 1 in which the resultant molecule is formed when anhydrous crystalline D-ribose is placed in the mouth.

20. The reaction according to claim 1 in which the resultant molecule is formed when one mole of D-ribose is mixed with one mole of water.

21. The reaction according to claim 1 in which the resultant molecule is formed before crystallization to remove the water is done in order to form the anhydrous crystalline, non-amorphous, near white commercial product.

22. The reaction according to claim 1 in which the resultant molecule is very soluble in water.

23. The reaction according to claim 1 in which the resultant molecule can be marketed as a concentrated aqueous solution.
